(19) United States
(12) Patent Application Publication
(10) Pub. No.: US 2016/0353973 A1
(43) Pub. Date: Dec. 8, 2016

(54) WIRELESS VIEWING DEVICE

(71) Applicant: A.M. SURGICAL, INC., Smithtown, NY (US)

(72) Inventors: Ather MIRZA, Smithtown, NY (US); Romi MIRZA, Smithtown, NY (US)

(21) Appl. No.: 15/227,463

(22) Filed: Aug. 3, 2016

Related U.S. Application Data
(63) Continuation-in-part of application No. 14/961,537, filed on Dec. 7, 2015.
(60) Provisional application No. 62/134,914, filed on Mar. 18, 2015.

(51) Int. Cl. A61B 1/00; A61B 17/34; A61B 1/06; A61B 1/317; A61B 1/05

(52) U.S. Cl. CPC A61B 1/00016; A61B 1/00108; A61B 1/317; A61B 1/05; A61B 1/0676; A61B 1/0684; A61B 1/00006; A61B 1/00154; A61B 17/3423; A61B 2017/320044

(57) ABSTRACT

The present application discloses a wireless device for performing endoscopic and arthroscopic procedures on any target tissue, bone, joint or target area of the body of a subject. The application also discloses a kit having the wireless device. A method for using the wireless device in endoscopic and arthroscopic procedures is also described and is applicable to any target tissue, bone, joint or target area of the body of a subject.

This application is a Continuation-In-Part application of U.S. patent application Ser. No. 14/961,537, filed on Dec. 7, 2015, which claims priority to Provisional U.S. Patent Application No. 62/134,914, filed on Mar. 18, 2015. The entirety of the aforementioned applications is incorporated herein by reference.

### FIELD

This application generally relates to medical devices. In particular, the application relates to wireless viewing devices and the use thereof.

### BACKGROUND

Conventional surgical techniques and equipment often require a fairly large incision over the surgical site and spreading of the incision to allow viewing and instrument access. These techniques can require a longer period of recovery than endoscopic methods and have greater levels of post-operative pain due to the incision size and level of manipulation during the procedure.

Endoscopic and arthroscopic surgeries are minimally invasive surgical procedures that are performed through small incisions or natural body openings. An endoscopic or arthroscopic procedure typically involves use of specialized devices and direct- or remote-control manipulation of instruments with indirect observation of the surgical field through an endoscope, arthroscope or similar device. Compared to open surgery, endoscopic and arthroscopic surgery is a minimally invasive surgery with less postoperative pain, early resumption of usual activities and a cosmetically appealing scar. It typically results in shorter hospital stays, or allows outpatient treatment.

In general, arthroscopy is applied to introduction of a scope into a joint anywhere in the body. Arthroscopic surgery refers to the process of introducing of the instrument to, and performing an operation at the joint. Endoscopy is applied to introduction of a scope into a body cavity anywhere in the body. Endoscopic surgery refers to the process of introducing the instruments and performing surgery at the operation site. Further nomenclature is designated by the anatomical structure the scope is introduced into, for example if the scope is placed in the stomach it is called Gastroscopy, in the abdomen it is Laprascopy, etc. There are places where no actual cavity exists. Here, surgeons can create a cavity by introducing a slotted cannula to visualize the surroundings without soft tissue obstruction, as in endoscopic carpal tunnel and cubital tunnel.

As seen in recent outbreaks of infections in hospitals, the insufficient or improper sterilization of re-usable surgical implements, can result in the introduction of microorganisms, including drug-resistant bacteria, into the patient, potentially resulting in severe, or even lethal, infections. This risk is magnified in procedures that require the insertion of multiple instruments into an incision.

### SUMMARY

The present application fulfills a need in the art for a compact, disposable device for uniportal endoscopic and arthroscopic surgery that comprises all of the individual tools needed, including a camera, following the creation of an entry portal. The device and method of the present application allow the insertion of a single member through the entry portal, with all of the individual tools contained there within, allowing that member to remain in situ throughout the procedure, thereby eliminating the need to insert and withdraw multiple instruments through the entry portal. The entire device can then be disposed of as medical waste following the procedure, thereby eliminating the need for re-sterilization and minimizing the threat of patient infection. Additionally, the present device comprises integrated camera and lighting, allowing wireless real-time transmission of images to any viewing device, providing the advantage of allowing small clinics or individual practitioners to provide endoscopic services without the need to invest in costly equipment that can also be expensive to maintain.

One aspect of the present application relates to a wireless viewing device for endoscopic and arthroscopic procedures. The device comprises a housing having a proximal end and a distal end; a wand having a proximal end and a distal end, wherein the proximal end of the wand adjoins the distal end of the housing; a camera with a lens; a light source; a control board having transmitter functions for wirelessly communicating with at least one external receiver; and a power source, wherein the control board and power source are enclosed by the housing and the camera with a lens and the light source are located at or proximate to the distal end of the wand. In some embodiments, the wireless viewing device is insertable into a cannula.

Another aspect of the present application relates to wireless device for performing video-assisted endoscopic and arthroscopic procedures. The device comprises a wand having a proximal end and a distal end; a camera with a lens; a light source for illuminating a target tissue; a control board having transmitter and receiver functions for wirelessly communicating with at least one external receiver/transmitter; an antenna; and a power source. The camera is located at the distal end of the wand, and the control board, light source and antenna are mounted inside an enclosed housing, wherein the housing has a distal end and a proximal end. The wand is attached to the distal end of the housing and the device further comprises a light-transmitting optical fiber extending from the proximity of the light source to the proximity of the distal end of the wand.

Still another aspect of the present application relates to a system for wireless endoscopic and arthroscopic procedures. The system comprises a wireless device and an external receiver/transmitter for visualizing images transmitted by the device. The device comprises a housing having a proximal end and a distal end; a wand having a proximal end and a distal end, wherein the proximal end of the wand adjoins the distal end of the housing; a camera with a lens; a light source; a control board having transmitter functions for wirelessly communicating with at least one external receiver; and a power source, wherein the control board and power source are enclosed by the housing and the camera with a lens and the light source are located at or proximate to the distal end of the wand.

Yet another aspect of the present application relates to a method for performing an endoscopic or arthroscopic procedure with a wireless viewing device. The device comprises a housing having a proximal end and a distal end; a wand having a proximal end and a distal end, wherein the proximal end of the wand adjoins the distal end of the housing; a camera with a lens; a light source; a control board having transmitter functions for wirelessly communicating with at least one external receiver; and a power source, wherein the control board and power source are enclosed by the housing and the camera with a lens and the light source are located at or proximate to the distal end of the wand. The method comprises the steps of: a) establishing an entry portal; b) inserting a distal end of a cannula through the entry portal; c) advancing the cannula to create a passage to a target tissue; d) inserting the distal end of the wand into the proximal end of the cannula and advancing the wand toward the distal end of the cannula; and e) imaging the target tissue with the camera.

A further aspect of the present application relates to a kit for performing an endoscopic or arthroscopic procedure. The kit comprises a wireless viewing device for endoscopic and arthroscopic procedures. The device comprises a housing having a proximal end and a distal end; a wand having a proximal end and a distal end, wherein the proximal end of the wand adjoins the distal end of the housing; a camera with a lens; a light source; a control board having transmitter functions for wirelessly communicating with at least one external receiver; and a power source, wherein the control board and power source are enclosed by the housing and the camera with a lens and the light source are located at or proximate to the distal end of the wand. In some embodiments, the kit further comprises a cannula.

### BRIEF DESCRIPTION OF THE DRAWINGS

The present application can be better understood by reference to the following drawings. The drawings are merely exemplary to illustrate certain features that may be used singularly or in any combination with other features and the present application should not be limited to the embodiments shown.

FIG. 1 shows an exemplary embodiment of the present application.

FIGS. 2A-C show top (2A), side (2B) and distal end (2C) perspective views of an exemplary embodiment of the present invention.

FIG. 3 shows an exemplary wireless viewing device of the present application.

### DETAILED DESCRIPTION

The following detailed description is presented to enable any person skilled in the art to make and use the object of this application. For purposes of explanation, specific nomenclature is set forth to provide a thorough understanding of the present application. However, it will be apparent to one skilled in the art that these specific details are not required to practice the subject of this application. Descriptions of specific applications are provided only as representative examples. The present application is not intended to be limited to the embodiments shown, but is to be accorded the widest possible scope consistent with the principles and features disclosed herein.

This description is intended to be read in connection with the accompanying drawings, which are to be considered part of the entire written description of this application. The drawing figures are not necessarily to scale and certain features of the application may be shown exaggerated in scale or in somewhat schematic form in the interest of clarity and conciseness. In the description, relative terms such as “front,” “back,” “up,” “down,” “top,” “bottom,” “upper,” “lower,” “distal,” and “proximal” as well as derivatives thereof, should be construed to refer to the orientation as then described or as shown in the drawing figure under discussion. These relative terms are for convenience of description and normally are not intended to require a particular orientation. Terms concerning attachments, coupling and the like, such as “connected,” “mounted,” and “attached,” refer to a relationship wherein structures are secured or attached to one another either directly or indirectly through intervening structures, as well as both movable or rigid attachments or relationships, unless expressly described otherwise.

The term “trigger finger,” as used herein, also refers to “trigger digit,” “trigger thumb,” and “stenosing tendovaginitis.”

As used herein, the terms “horizontal” and “vertical,” and derivatives of those terms, are used in respect to their relationship to the plane defined by the slot in the cannula of the present application. “Vertical” refers to the plane that can, for example, pass through the slot of the cannula and bisect the cannula into two equal halves, while “horizontal” refers to a plane that is perpendicular to the vertical plane. The horizontal plane may be a level plane with respect to the length of the cannula or housing of the device, or may be at an angle to that level plane, allowing some upward or downward movement of elements moving along the horizontal plane with respect to the level plane.

As used herein, the term “subject” refers to an animal. In some embodiments, the animal is a mammal. In further embodiments, the mammal is a human.

The present application describes a disposable, compact device for performing uniportal endoscopic and arthroscopic surgical procedures. In some embodiments, the device comprises a rigid or flexible cannula, dependent upon the procedure for which the device is to be used, that is attached to the distal end of a housing. In some further embodiments, the cannula is clear, being made of a transparent material. In some embodiments, the procedure can be a uniportal percutaneous endoscopic surgical procedure.

In some embodiments, the cannula has an open proximal end where it is attached to the distal end of the housing, such that surgical tools stored within the housing can be extended into the cannula. In some embodiments, the cannula comprises a closed distal end. In some further embodiments, the closed distal end of the cannula is upturned and comprises an edge for separating, but not cutting, tissues as the cannula is advanced from an entry portal towards a target tissue. In some embodiments, the cannula comprises a longitudinal slot that extends from the proximate end to the proximity of the distal end of the cannula. In some further embodiments, the distal end of the slot is contiguous with an open distal end of the cannula. In other further embodiments, the distal end of the slot is closed. In still other embodiments, the cannula comprises open proximate and distal ends, with the longitudinal surfaces of the cannula being closed.

The housing further comprises a blade and camera that can be extended into the cannula. Particular embodiments of the present device also include a probe and/or cautery within the housing and that can be extended into the cannula.

Additional embodiments of the present invention may include any type of tool that would be useful for a general or specific endoscopic or arthroscopic surgical procedure, such as a rasp for clearing synovium or implements for harvesting a section of tissue for biopsy, grafting or ex vivo growth. The device also comprises a mechanism for advancing/withdrawing the aforementioned tools into/from the cannula. Tools such as the blade, probe and cautery are advanced into the cannula along with the camera in order to perform a desired procedure. The tool protrudes vertically through the slot of the cannula in order to contact a target tissue and perform the desired procedure, while under the observation of the camera, which remains within the lumen of the cannula. Additionally, the housing further comprises a light source for illuminating said procedure, a circuit board for controlling the functions of the device, a wireless transmitter/receiver/antenna assembly for communicating between the device and a remote control panel or video monitor. The housing further contains a power source, such as a battery. The preassembled nature of the device also provides convenience for the practitioner in that the cannula, camera and tools are available in a single package that requires no further assembly and can be used easily in an office setting without the need for some traditional endoscopic or arthroscopic equipment that may be too expensive or cumbersome to use outside of a hospital. Additionally, the present device also can be easily transported and used in remote settings, such as by emergency medical personnel, first responders or military medical personnel.

The use of the present device is exemplified in this application for, but not limited to, endoscopic surgical division of a pulley or tunnel. Some other non-limiting uses for the present device include, for example, other divisions or partial separation of a tendon or ligament, cutting, dividing, separating or making an incision in connective tissue, muscle, cartilage, membranes, skin, other body tissues or organs or any other use of the device that can be envisioned or carried out by the practitioner. As used herein, the term “practitioner” refers to one of skill in the art or any other user of the present device. In some embodiments, the device can be used for a uniportal endoscopic viewing and/or surgical procedure. In other embodiments, the device can be used for an arthroscopic, laparoscopic, or thoracoscopic viewing and/or surgical procedure. As used herein, “laparoscopic” and “thoracoscopic” procedures fall within the scope of “endoscopic” and “arthroscopic” procedures.

Endoscopic or arthroscopic surgical procedures that can be performed with a cannula or device of the present application include, but are not limited to, carpal tunnel release, Guyon's canal (or tunnel) release, cubital tunnel release, plantar fascia release, lateral release for patella realignment, release of radial tunnel, release of pronatar teres, release of trigger finger, release of lacertus fibrosus, tendon release, release of the extensor tendons for lateral epicondylitis, release of medial epicondylitis, release of the posterior and other compartments of the leg, forearm fascia release for fascial compartment syndrome, release of fascial compartments in the upper or lower extremities, relieving the compression of a nerve by a ligament pulley or tunnel, and releasing the travel of a ligament or tendon through a pulley or tunnel. Procedures that can be performed with a cannula or device of the present application include endoscopic or arthroscopic surgical procedures on the spine, such as discectomy for the treatment of degenerative disc disease, herniated discs, bulging discs, pinched nerves or sciatica. Procedures that can be performed with a cannula or device of the present application also include procedures on cranial and facial tissues, as well as fasciotomy release throughout the body. The cannula or device of the present application can be used for blood vessel, including vein or artery, harvesting throughout the body, for example to provide blood vessel graft material in conjunction with a coronary bypass procedure or for a reconstructive surgical procedure. Procedures that can be performed with a cannula or device of the present application also include endoscopic procedures on the wrist and hand, including the palmar and dorsal sides of the hand. Endoscopic procedures that can be performed with a cannula or device of the present application on the hand also include the digits, including the thumb, index finger, middle finger, ring finger and little (pinky) finger. Other examples of endoscopic or arthroscopic procedures that can be performed with a device of the present application include, but are not limited to, observation of internal tissues or injuries, cauterization of vessels, harvesting of tissues for ex vivo growth; obtaining biopsies; spinal surgery; endonasal surgery; mucosal resection; removal of parasites, cysts or tumors, and foreign body retrieval. Still other examples of endoscopic or arthroscopic surgery that can be performed with the device include, but are not limited to, procedures on or within bone, in or around joints or the tendons associated with those joints, as well as any tissue, area or cavity of the body of a subject.

In some embodiments, the present device can be used in the head of a subject. Exemplary procedures in the head include, but are not limited to, nasal surgery, endoscopic sinus surgery, endoscopic pituitary surgery, cranial surgery, endoscopic ear surgery, throat surgery, endodontic surgery and tonsils.

In some embodiments, the present device can be used in the neck of a subject. Exemplary procedures in the neck include, but are not limited to, laryngoscopic surgery, vocal cord surgery, esophageal surgery, thyroid surgery, carotid artery surgery, and brachial plexus surgery.

In some embodiments, the present device can be used in the chest of a subject. Exemplary procedures in the chest include, but are not limited to, endoscopic mediastinal surgery, thoracic surgery, heart surgery, esophageal surgery, and upper gastrointestinal (GI) scoping.

In some embodiments, the present device can be used in a procedure of a finger, hand, foot of a subject.

In some other embodiments, the present device can be used in the abdomen of a subject. Exemplary procedures in the abdomen include, but are not limited to, diagnostic laparoscopy, laparoscopic gastric surgery, laparoscopic liver surgery, laparoscopic pancreatic surgery, laparoscopic nephrectomy and kidney surgery, laparoscopic intestinal surgery, laparoscopic oophorectomy, laparoscopic hysterectomy, laparoscopic urinary bladder surgery, laparoscopic prostate surgery, laparoscopic aortic surgery, laparoscopic appendectomy, laparoscopic colon surgery, endoscopic hysterotomy, endoscopic fetal surgery, endoscopic hernia repair, and endoscopic splenectomy.

In some embodiments, the present device can be used in an upper extremity of a subject. Exemplary procedures in an upper extremity include, but are not limited to, ECTR, ECUTR, endoscopic pronator teres release, forearm fascial compartment release, endoscopic repair of biceps tendon, endoscopic release of lateral and medial epicondylitis, endoscopic release of radial tunnel syndrome, endoscopic surgery of the brachial plexus, endoscopic harvesting of nerve graft, arthroscopy and surgery of wrist, arthroscopy of elbow, arthroscopy and surgery of the carpometacarpal (CMC) joint, arthroscopy and surgery of shoulder, arthroscopy and surgery of acromioclavicular (AC) joint.

In some embodiments, the present device can be used in a lower extremity of a subject. Exemplary procedures in an lower extremity include, but are not limited to, femoral artery surgery, fascia lata release, knee lateral release, endoscopic peroneal nerve release, endoscopic leg fascial compartment release, endoscopic release of gastrocnemius, endoscopic tarsal tunnel release, endoscopic release of Morton's neuroma, endoscopic release of the plantar fascia, arthroscopy of hip, knee and ankle, subtalar joint, and endoscopic harvesting of nerve and tendon graft.

Endoscopic or arthroscopic surgical procedures that can be performed with a device of the present application, such as, but not limited to, a ligament or fascia release procedure, can be performed by approaching the target tissue through an incision or body opening on either the proximate or distal side of the target tissue.

In some embodiments, a device of the present application can be used for plastic surgery. A device of the present application is useful for tissue remodeling or the excision of tissue segments, including necrotic tissue.

A device of the present application is lightweight, compact and can be manipulated with a single hand. The weight of the device is less than about one pound, allowing the device to be easily carried within a pocket, backpack, satchel or case. In some embodiments, the device weighs less than about 15, 14, 13, 12, 11, 10, 9, 8, 7, 6, 5, 4 or 3 ounces.

The housing of the device can be generally rectangular or oval in shape. The housing can also serve as a grip or handle for the device. All on-board components of the device are housed within a single housing, or within the housing and the cannula. In general, the housing of the device can be easily held within the palm of one hand and manipulated by that one hand. In some embodiments, the overall dimensions of the housing (with the longest dimension being measured from the distal end to the proximal end of the housing) are less than about 7 inches in length, 2 inches in width and 2 inches in thickness (7×2×2). In further embodiments, the overall dimensions of the housing are less than about 6×2×1. In still further embodiments, the overall dimensions of the housing are less than about 5×1.5×1. In even further embodiments, the overall dimensions of the housing are less than about 5×1.5×0.5. The outer surface of the housing may be textured, grooved, indented or shaped to facilitate gripping by a hand or by another device.

The cannula of the device is attached at its proximal end to the distal end of the housing. The cannula can be either permanently attached to the distal end, or may be detachable/replaceable by any suitable means including, but not limited to, a luer-lock type system, cam lock, snap-fit or threaded to screw into or onto the distal end of the housing. The cannula can be opaque, translucent or transparent. The cannula can be flexible or rigid. In some embodiments, the cannula is made of polycarbonate. In some embodiments a cannula of the present device is about 12 inches in length. In some embodiments, a cannula of the present device is about 11, 10, 9, 8, 7, 6, 5, 4, 3 or 2 inches in length. In some embodiments, the cannula is a rigid, transparent, cylinder-shaped tube with an open slot extending from its proximal end or the proximity of its proximal end to its distal end or the proximity of its distal end.

In some embodiments, one or more detachable cannulas may be supplied with a housing as part of a kit. Each cannula supplied with the housing may be designed or suited for a particular need for the examination and/or treatment of a subject. For example, in a kit with multiple cannulas supplied, the practitioner can select a cannula that is best suited for use with the immediate treatment/examination need of the subject. The cannulas can be interchanged during the treatment of that subject based upon changing needs by the practitioner for the treatment/examination of that subject.

The compact size and light weight of the device reduces the amount of fatigue experienced by the practitioner operating the device versus larger, heavier devices.

The device can be supplied as a single-use, disposable device that is pre-sterilized and sealed within packaging that keeps the device sterile until opened. The device can be supplied as part of a kit that includes additional instruments useful with the device such as, but not limited to, scalpel, elevator, dilator, bandages, tape, needles and sutures.

The device can be used in a clinical setting. The clinical setting can be a hospital, emergency clinic, outpatient clinic, or office, for example. The device can also be used outside the clinical setting, such as, but not limited to, in an emergency situation. The device of the present application can be used by various practitioners including, but not limited to, a physician, surgeon, nurse, nurse practitioner, first responder, paramedic, emergency medical technician, medic, corpsman, technician or caregiver.

One aspect of the present application relates to a device for endoscopic and arthroscopic procedures. The device comprises a housing having a proximal end and a distal end; a cannula having a proximal end and a distal end, wherein the proximal end of the cannula adjoins the distal end of the housing; a camera with a lens; a light source; a control board having transmitter functions for wirelessly communicating with at least one external receiver; and a power source, wherein the control board and power source are enclosed by the housing.

In some embodiments, the device can be used on any target tissue, bone, joint or target area of the body of a subject.

In particular embodiments, the device can be handheld.

In some embodiments, the camera is enclosed in the housing.

In other embodiments, the camera is located in the proximity of a distal end of the cannula.

In some embodiments, the device further comprises an antenna.

In other embodiments, the device further comprises a receiver for receiving wireless communication from an external transmitter.

In particular embodiments, the cannula is made of a transparent material.

In some embodiments, the distal end of the cannula comprises an edge for separating tissues.

In other embodiments, the distal end of the cannula is turned upwards like an obturator.

In some embodiments, the lens of the camera has a diameter of less than 5 mm.

In particular embodiments, the light source is an LED light source.

In other embodiments, the camera is a NANEYE camera.

In some embodiments, wireless communication is performed by Bluetooth, radio frequency, or WiFi.

Another aspect of the present application relates to a wireless device for performing video-assisted endoscopic and arthroscopic procedures. The device comprises a cannula having a tubular body with a proximal end and a distal end; a camera with a lens; a light source that illuminates into and through the cannula; a control board having transmitter and receiver functions for wirelessly communicating with at least one external receiver/transmitter; an antenna; and a power source, wherein the light source, control board, power source, and antenna are mounted inside a single enclosed housing, wherein the housing has a distal end and a proximal end and wherein the cannula is attached to the distal end of the housing.

In some embodiments, the device can be used on any target tissue, bone, joint or target area of the body of a subject.

In particular embodiments, the device can be handheld. In further embodiments, the housing of the device can be held in the palm of one hand.

In some embodiments, the camera is enclosed in the housing.

In other embodiments, the camera is located in the proximity of a distal end of the cannula.

In some embodiments, the cannula is flexible.

Still another aspect of the present application relates to a system for wireless endoscopic and arthroscopic procedures, comprising a wireless device and an external receiver/transmitter for visualizing images transmitted by the device. The device comprises a housing having a proximal end and a distal end; a cannula having a proximal end and a distal end, wherein the proximal end of the cannula adjoins the distal end of the housing; a camera with a lens; a light source; a control board having transmitter functions for wirelessly communicating with at least one external receiver; and a power source, wherein the control board and power source are enclosed by the housing.

In some embodiments, the device can be used on any target tissue, bone, joint or target area of the body of a subject.

In particular embodiments, the external wireless receiver/transmitter is selected from the group consisting of video monitors, computer terminals comprising a monitor, smart phones and tablet computers.

Yet another aspect of the present application relates to a method for performing an endoscopic or arthroscopic procedure with a wireless device. The device comprises a housing having a proximal end and a distal end; a cannula having a proximal end and a distal end, wherein the proximal end of the cannula adjoins the distal end of the housing; a camera with a lens; a light source; a control board having transmitter functions for wirelessly communicating with at least one external receiver; and a power source, wherein the control board and power source are enclosed by the housing. The method comprises the steps of: a) establishing an entry portal; b) inserting the distal end of the cannula through the entry portal; c) advancing the cannula to create a passage to a target tissue; and d) imaging the target tissue with the camera.

In some embodiments, the device can be used on any target tissue, bone, joint or target area of the body of a subject.

A further aspect of the present application relates to a kit for performing an endoscopic or arthroscopic procedure, comprising a wireless device and instructions. The device comprises a housing having a proximal end and a distal end; a cannula having a proximal end and a distal end, wherein the proximal end of the cannula adjoins the distal end of the housing; a camera with a lens; a light source; a control board having transmitter functions for wirelessly communicating with at least one external receiver; and a power source, wherein the control board and power source are enclosed by the housing.

In some embodiments, the device can be used on any target tissue, bone, joint or target area of the body of a subject.

In some embodiments, the kit further comprises an instrument for establishing an entry portal.

In other particular embodiments, the camera views the tissues surrounding the cannula. In further embodiments, the camera is extendable into the cannula along with the probe, blade, or cautery. In still further embodiments, the camera views the probe, camera, or cautery. In other still further embodiments, the extension of the camera is controlled remotely.

In particular embodiments, the view of the camera can be rotated.

In some embodiments, the camera lens has a diameter of less than about 5 mm. In still other embodiments, the camera lens has a diameter of less than about 4 mm. In yet other embodiments, the camera lens has a diameter of less than about 3 mm. In even other embodiments, the camera lens has a diameter of less than about 2 mm.

In other particular embodiments, the camera is a NANEYE camera.

In some embodiments, the camera lens is an optical fiber lens.

In some embodiments, the light source is an LED light source.

In some embodiments, the light source has a diameter of less than about 5 mm. In still other embodiments, the light source has a diameter of less than about 4 mm. In yet other embodiments, the light source has a diameter of less than about 3 mm. In even other embodiments, the light source has a diameter of less than about 2 mm.

In some embodiments, the light source is attached to a fiber optic cable for transmitting light in the cannula. In some further embodiments, the fiber optic cable is embedded into a wall of the cannula. In some embodiments, the fiber optic cable illuminates the entire length of the cannula.

In still other embodiments, the cannula is expandable laterally to spread tissue.

In yet other embodiments, the power source is a battery.

In some particular embodiments, the device is single-use and disposable.

In some embodiments, the device comprises an information display provided in the housing to display frequency information of a transmission frequency of the wireless transmitter. In further embodiments, the device comprises an information reading device for reading the frequency information of the information display.

In some embodiments, the internal control board having transmitter and receiver functions for wirelessly communicating and the at least one external receiver and transmitter communicate via radio frequency communication signals (e.g., FM radio signal). In other embodiments, communication is via microwave or infrared (IR) communication signals from the wireless sensor. In still other embodiments, communication is via short-wavelength radio transmissions, for example in the ISM band from 2400-2480 MHz (IEEE 802.15.1, or Bluetooth). In even other embodiments, communication is via near-field communication (NFC) signals between the internal control board having transmitter and receiver functions for wirelessly communicating and the at least one external receiver and transmitter.

In some embodiments, the external receiver and transmitter is interfaced with a computer terminal. In some further embodiments, the computer terminal is a notebook computer. In other embodiments, the external receiver and transmitter is interfaced with a tablet or smart phone.

In some embodiments, each of said tools is individually associated with an independent camera operatively attached to that tool.

In other embodiments, the housing further comprises a video display showing real time images from a camera of the device.

In some embodiments, the device comprises non-volatile memory for storing images or information from a performed procedure, wherein said images or information can be retrieved from the device. In some embodiments, the non-volatile memory is an RFID tag. In other embodiments, the non-volatile memory is a micro-SD card.

In some embodiments, the device could be attached to, held, by, inserted into or under the control of a robotic device or tool.

In some embodiments, the device can be directly controlled such as, but not limited to, manual control. In other embodiments, the device can be remotely controlled.

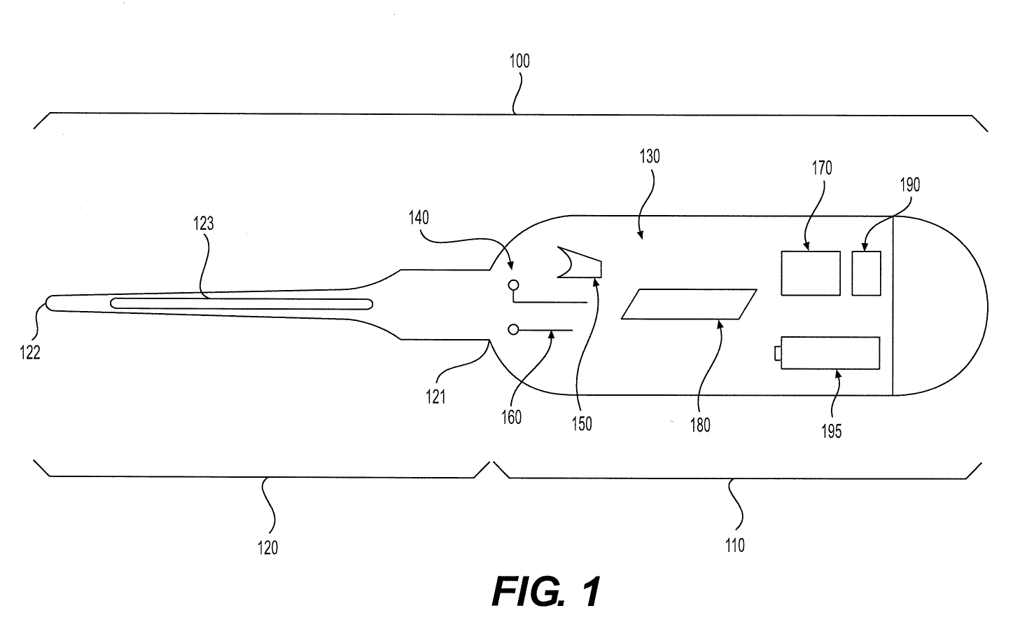

FIG. 1 shows one exemplary embodiment of the device 100 of the present application. The device generally comprises a housing 110 and a cannula 120. The cannula 120 comprises an open central lumen, a proximal end 121 and a distal end 122, wherein the proximal end 121 of the cannula 120 is attached to the distal end of the housing 110.

In some embodiments, the cannula 120 is composed of a clear material. In further embodiments, the clear material is polycarbonate. In some embodiments, the cannula is marked with gradations showing how far the cannula 120 had been inserted through an entry portal.

The cannula 120 of the present device 100 comprises a slot 123 in its upper surface, wherein said slot 123 is contiguous with the open central lumen. In some embodiments, said upper surface is flattened, in other embodiments, said upper surface is rounded. In some embodiments, the slot 123 extends from the proximal end 121 to the proximity of the distal end 122 of the cannula 120. In other embodiments, the slot extends from a point located between the proximal end 121 and distal end 122 to the proximity of the distal end 122. As used herein, “the proximity of the distal end” has the meaning of the slot 123 ending prior to actually joining the distal end 122, i.e., having at least a minimal bridge of material crossing between the distal end of the slot 123 and the distal end 122 of the cannula 120 to prevent over-advancement of a deployed tool through the distal end 122 of the cannula 120.

In other embodiments, the distal end of the slot 123 is contiguous with an open distal end 122 of the cannula 120.

In some embodiments, the distal end 122 of the cannula 120 is closed, as an obturator. In some embodiments, said distal end 122 is pointed. In other embodiments, the distal end 122 comprises a leading edge that is turned upwards, allowing the cannula to separate and form a passage from the entry portal through/between/under/over body tissues to and/or past a target tissue. In some further embodiments, the edge can be flattened.

In some embodiments, the body of the cannula 120 is laterally expandable in order to spread tissue as a passage is made by the cannula 120, obviating the need for inserting a separate instrument through the entry portal to spread tissue.

Also as shown in FIG. 1, the camera 130 and surgical tools are contained within the housing 110 of the device 100 prior to deployment into the cannula 120.

The device 100 comprises a camera 130 that is small enough to deploy into the central lumen of the cannula 120. The camera 130 is generally a high resolution camera, but is at least of sufficient resolution for imaging with sufficient clarity to distinguish different bodily tissues from one another and to image a target tissue with sufficient clarity in order to observe the performance of a surgical procedure on the target tissue. In some embodiments, the camera 130 can be focused The camera 130 can be advanced into the cannula 120 independently of any surgical tools in order to image/observe bodily tissues or target tissue surrounding the cannula 120 or through the slot 123 before, after or in lieu of a surgical procedure. Having an integral camera 130 within the device, eliminates the need to insert a separate endoscopic camera into the device or an entry portal, thereby eliminating the need for another separate element in the procedure.

The camera 130 can also be advanced into the cannula 120 in association with the probe 140, blade 150, cautery 160 or other suitable surgical tool. In general, the camera comprises within its field of view any portion of the probe 140, blade 150, cautery 160 or other suitable surgical tool that is in contact with, or performing a desired surgical procedure on, a target or bodily tissue. In some embodiments, the camera is a NANEYE camera. In other embodiments, the camera 130 has a resolution of at least 100×100 pixels. In a further embodiment, the camera 130 has a resolution of at least 150×150 pixels. In a still further embodiment, the camera 130 has a resolution of at least 200×200 pixels. In an even further embodiment, the camera 130 has a resolution of at least 250×250 pixels. In some embodiments, there is a separate camera 130 independently associated with each tool of the device 100.

In another embodiment, the camera 130 remains in a fixed position within the housing 110. In a further embodiment, the camera 130 comprises an image transmitting optical fiber, which is attached at its proximal end to the camera. In some still further embodiments, the distal end of the image transmitting optical fiber is movable and moves into the cannula 120 independently or with tools of the device 100. In other still further embodiments, the distal end of the image transmitting optical fiber is in a fixed position in the proximity of the distal end 122 of the cannula, such that from the fixed position of the image transmitting optical fiber the camera 130 can observe and image the surgical procedure.

In still another embodiment, the device 100 comprises a combination of at least one movable camera 130 and at least one fixed position camera 130 as described above. In some embodiments, a camera 130 of the present device 100 comprises a camera body and a lens assembly that is attached to the camera body via an image transmitting optical fiber.

Still referring to FIG. 1, in some embodiments, the device comprises a probe element 140. The probe 140 can be advanced into the cannula 120 and protrudes vertically through the slot 123 in order to, for example, move tissues above the slot 123, provide a reference point for imaging, determine the edges of the target tissue or remove synovium from the target tissue.

Also referring to FIG. 1, the device 100 comprises a blade 150 for performing surgical procedures on a target tissue. The blade 150 can be advanced into the cannula 120 and protrudes vertically through the slot 123 in order to divide a target tissue. In some embodiments, the blade 150 comprises at least one cutting surface on its distal side and division of the target tissue is performed by moving the blade through the slot 123 in a proximal 121 to distal 122 direction. In other embodiments, the blade 150 comprises at least one cutting surface on its proximal side and division of the target tissue is performed by moving the blade through the slot 123 in a distal 122 to proximal 121 direction.

FIG. 1 also shows an embodiment of the device 100 comprising a cautery element 160. The cautery 160 can be advanced into the cannula 120 and protrudes vertically through the slot 123 in order to cauterize a target tissue. In some embodiments, the target tissue was previously divided with the blade 150 of the device 100 during the same surgical procedure. In another embodiment, the target tissue was previously divided by a blade in an earlier surgical procedure. In yet another embodiment, the target tissue was in need of cauterizing due to an earlier injury or other outstanding medical condition.

In some embodiments, the device 100 further comprises a light source 170 contained within the housing 110. The light source 170 provides illumination for the camera 130 in order to allow visualization of bodily or target tissues through the cannula 120 or slot 123. In some embodiments, the position of the light source 170 is fixed within the housing 110 of the device 100. In other embodiments, the light source 170 is associated with the camera 130 and travels with the camera 130, either into the cannula 120, or staying within the housing 110, but moving closer to the proximal end 121 of the cannula 120 as the camera 130 is advanced toward the distal end 122 of the cannula 120. In still other embodiments, the light source 170 comprises a main body whose position is fixed within the housing and is attached to the proximal end of a light transmitting fiber, the distal end of which provides light for the camera 130. In some further embodiments, the distal end of the light transmitting fiber moves in concert with the camera 130. In other further embodiments, the distal end of the light transmitting fiber remains in a fixed position within the cannula 120, for example, in the proximity of the distal end 122 of the cannula. In some embodiments, the light source 170 is a semiconductor light source. In some embodiments, the light source 170 is a light emitting diode (LED) light source. In some embodiments, the device 100 comprises a plurality of light sources 170 as described above that can be in a fixed position and/or moveable.

Still in FIG. 1, in some embodiments, the device 100 comprises a circuit board 180 for processing imagery obtained by the camera 130. Said imagery is transmitted to a remote control or video display via a wireless antenna 190 contained within the housing 110 of the device 100. In some instances, the circuit board receives instructions from the remote control via the wireless antenna 190. In some embodiments, the movement of the camera 130 and the tools of the device 100 are controlled remotely via instruction transmitted to the circuit board 180. In other embodiments, the housing 110 of the device 100 comprises manual control for selecting tools and/or advancing/withdrawing tools or the camera 130 into/from the cannula 120.

Also depicted in FIG. 1, the device 100 further comprises an integral power source 195 to provide energy for the camera 130, light source 170, circuit board 180 and any mechanical functions within the device 100. In some embodiments, the power source 195 is a battery. In further embodiments, the battery is a lithium battery. In some embodiments, the power source 195 is installed within the device 100 upon manufacture, or prior to provision to a practitioner. In other embodiments, the power source 195 is provided separately from the device 100 and installed into the device 100 prior to the use of the device 100 in a surgical procedure. In some embodiments, the power source 195 is removable from the device 100 for separate disposal.

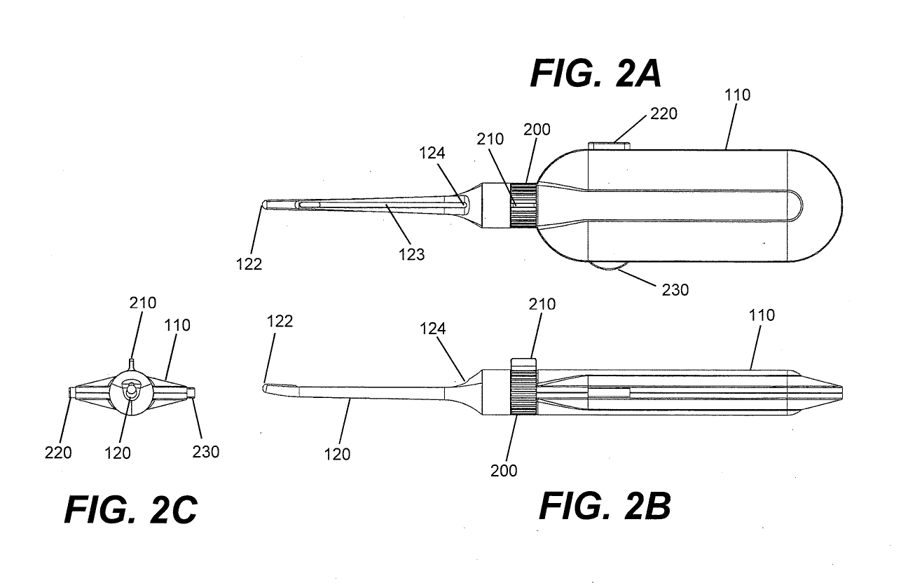

FIG. 2A shows a top view of an embodiment of the device, showing a cannula 120 attached at its proximal end to the distal end of a housing 110. In this embodiment, the cannula 120 comprises a longitudinal slot 123 that extends longitudinally from a proximal end 124 to near the distal end 122 of the cannula 120. In some embodiments, the proximal end 124 of the slot is raised to better allow tools to enter the slot without scraping against the proximal end 124. In some embodiments, at the junction of the housing 110 and the cannula 120, the device comprises a connecting ring 200 that attaches the cannula 120 to the housing 110. In some embodiments, the connecting ring 200 comprises a paddle 210 for ease of manipulation, such as when the device is being held and controlled within one hand. In some embodiments, the connecting ring allows for the interchangeable attachment of different types of cannulas 120 to a given housing 110, dependent upon the procedure to be performed with the device. In some embodiments, the housing 110 further comprises a rocker switch 220 that is used to move the selected tools within the housing or into and out of the cannula 120. In other embodiments, the movement of tools into and out of the cannula 120 is controlled electronically and/or remotely. In some embodiments, the housing 110 also comprises a switch 230 for turning the light on and off.

FIG. 2B is a side view of the embodiment of the device shown in FIG. 2A, showing the housing 110, cannula 120, and the intervening connecting ring 200 with paddle 210. In some embodiments, the distal end 122 of the cannula 120 is closed and turned upwards, serving as an integral obturator.

FIG. 2C is a distal end view of the embodiment of the device shown in FIG. 2A, showing the relative positions of the cannula 120, housing 110, paddle 210, slider knob 220 and switch 230.

Another aspect of the present application relates to a wireless viewing device for observation of bodily tissues. The wireless viewing device comprises a housing having a proximal end and a distal end; a wand having a proximal end and a distal end, wherein the proximal end of the wand adjoins the distal end of the housing; a camera with a lens; a light source; a control board having transmitter functions for wirelessly communicating with at least one external receiver; and a power source, wherein the control board and power source are enclosed by the housing and the camera with a lens and the light source are located at or proximate to the distal end of the wand. In some embodiments, the wireless viewing device is insertable into a cannula.

As with other embodiments of the present application, the wireless viewing device of this embodiment can be used on any target tissue, bone, joint or target area of the body of a subject as set forth above. In some embodiments, the wireless viewing device of this embodiment can be used for minimally-invasive observation of an internal bodily target location. The wireless viewing device can be used, for example, for pre-treatment, or pre-operative, observation or diagnosis; post-treatment, or post-operative, observation or follow-up; or general observation of a target location in need thereof without further treatment or operative intervention. In some embodiments, the wireless viewing device of this embodiment can be used for monitoring of the progress or regression of a condition or target tissue, such as a surgical repair of a target tissue or the growth/regression of a neoplasm or tumor. The device comprises a housing having a proximal end and a distal end; a wand having a proximal end and a distal end, wherein the proximal end of the wand adjoins the distal end of the housing; a camera with a lens; a light source; a control board having transmitter functions for wirelessly communicating with at least one external receiver; and a power source, wherein the control board and power source are enclosed by the housing and the camera with a lens and the light source are located at or proximate to the distal end of the wand. In some embodiments, the wireless viewing device is insertable into a cannula.

A wireless viewing device as embodied by the present application has an advantage for the practitioner and the subject in that it can be provided sterile as a single use and disposable device, without the need to be physically attached to monitoring or viewing equipment, thereby eliminating the possibility of transmitting infectious agents between subjects, which is a risk with reusable instruments that must be sterilized between uses. Additionally, the wireless viewing device can be used with a variety of devices that traverse an entry portal into the body of a subject for observation of a target tissue. Such devices that traverse an entry portal include, but are not limited to, a cannula, anoscope, port or any other suitable tubular entry device. In some embodiments, both the wireless viewing device and the device that traverses an entry portal are single use and disposable, further enhancing the elimination of the possibility of transmitting infectious agents between subjects.

The wireless viewing device described herein is completely self-contained within the singular housing and wand. The present device eliminates the need for connectors or attachments such as external pods comprising additional power sources, light sources, transmitters & receivers or imaging equipment. This elimination of the need for additional components simplifies the use for the practitioner, as well as enhances the sterility of the device.

A wireless viewing device of the present application is lightweight, compact and can be manipulated with a single hand. The weight of the device is less than about one pound, allowing the device to be easily carried within a pocket, backpack, satchel or case. In some embodiments, the device weighs less than about 15, 14, 13, 12, 11, 10, 9, 8, 7, 6, 5, 4 or 3 ounces.

The housing of the wireless viewing device can be generally rectangular or oval in shape. The housing can also serve as a grip or handle for the device. All on-board components of the device are housed within a single housing, or within the housing and the wand. In general, the housing of the device can be easily held within the palm of one hand and manipulated by that one hand. In some embodiments, the overall dimensions of the housing (with the longest dimension being measured from the distal end to the proximal end of the housing) are less than about 7 inches in length, 2 inches in width and 2 inches in thickness (7×2×2). In further embodiments, the overall dimensions of the housing are less than about 6×2×1. In still further embodiments, the overall dimensions of the housing are less than about 5×1.5×1. In even further embodiments, the overall dimensions of the housing are less than about 5×1.5×0.5. The outer surface of the housing may be textured, grooved, indented or shaped to facilitate gripping by a hand or by another device.

The wand of the wireless viewing device is attached at its proximal end to the distal end of the housing. The wand can be either permanently attached to the distal end, or may be detachable/replaceable by any suitable means including, but not limited to, a luer-lock type system, cam lock, snap-fit or threaded to screw into or onto the distal end of the housing. The wand can be opaque, translucent or transparent. The wand can be flexible or rigid. In some embodiments, the wand is made of polycarbonate. In some embodiments a wand of the present device is about 12 inches in length. In some embodiments, a wand of the present device is about 11, 10, 9, 8, 7, 6, 5, 4, 3 or 2 inches in length.

In some embodiments, one or more detachable wands may be supplied with a housing as part of a kit. Each wand supplied with the housing may be designed or suited for a particular need for the examination and/or treatment of a subject. For example, in a kit with multiple wands supplied, the practitioner can select a wand that is best suited for use with the immediate treatment/examination need of the subject. The wands can be interchanged during the treatment of that subject based upon changing needs by the practitioner for the treatment/examination of that subject.

The wireless viewing device can be supplied as a single-use, disposable device that is pre-sterilized and sealed within packaging that keeps the device sterile until opened. The device can be supplied as part of a kit that includes additional instruments useful with the device such as, but not limited to, scalpel, elevator, dilator, bandages, tape, needles and sutures.

The wireless viewing device can be used in a clinical setting. The clinical setting can be a hospital, emergency clinic, outpatient clinic, or office, for example. The device can also be used outside the clinical setting, such as, but not limited to, in an emergency situation. The device of the present application can be used by various practitioners including, but not limited to, a physician, surgeon, nurse, nurse practitioner, first responder, paramedic, emergency medical technician, medic, corpsman, technician or caregiver.

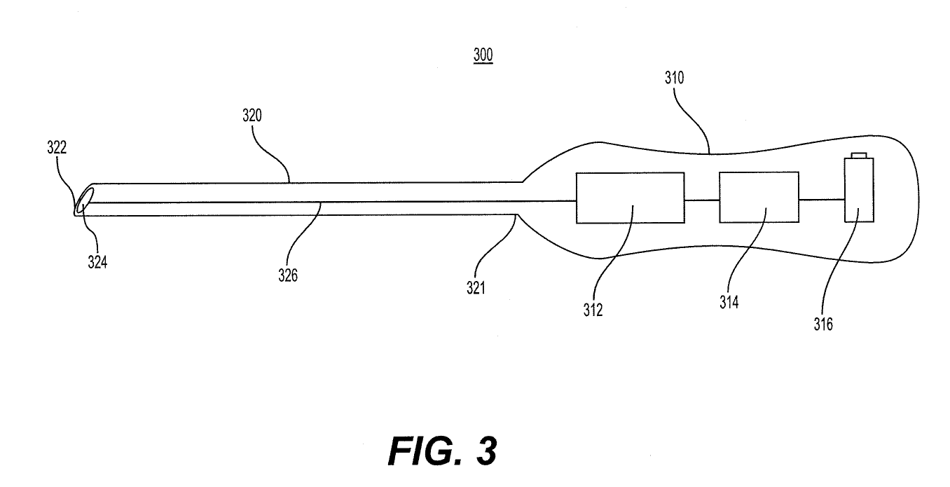

FIG. 3 illustrates an exemplary wireless viewing device 300 of the present application. The wireless viewing device comprises a housing 310 that also serves as a handle for the device and a wand 320, the proximal end of which is attached to the distal end of the housing 310. In some embodiments, the device can be handheld. In some further embodiments, the housing is formed in a shape to facilitate gripping with a single hand. In some embodiments, the device can be directly controlled such as, but not limited to, manual control. In other embodiments, the device can be remotely controlled. In some embodiments, the device can be held by another device. In some embodiments, the device could be attached to, held, by, inserted into or under the control of a robotic device or tool. In some embodiments, a light source 312 is contained within the housing 310. In some embodiments, the light source 312 is an LED light source. In some embodiments, the light source 312 shines through an opening in the distal end of the housing that is contiguous with the central lumen of the wand 320 to provide illumination for the camera 324. In other embodiments, the light source 312 illuminates a fiber optic 326 that extends into the wand 320. In still other embodiments, the light source 312 is located at the distal end of the wand 320.

Still referring to FIG. 3, the housing 310 further contains an internal control board having transmitter functions 314 that is capable of, for example, sending video images obtained by the wireless viewing device 300 to least one external receiver. In some embodiments, the device comprises an information display provided in the housing 310 to display frequency information of a transmission frequency of the wireless transmitter. In some embodiments, the internal control board has receiver functions, such that functions of the wireless viewing device can be controlled remotely. For example, functions that could be controlled remotely include, but are not limited to, camera focus, camera optical zoom, camera digital zoom, camera field of view, camera angle, camera rotation, light on/off, and light intensity.

In some embodiments, the internal control board having transmitter functions 314 for wirelessly communicating and the at least one external receiver communicate via radio frequency communication signals (e.g., FM radio signal). In other embodiments, communication is via microwave or infrared (IR) communication signals from the wireless sensor. In still other embodiments, communication is via short-wavelength radio transmissions, for example in the ISM band from 2400-2480 MHz (IEEE 802.15.1, or Bluetooth). In even other embodiments, communication is via near-field communication (NFC) signals between the internal control board having transmitter and receiver functions for wirelessly communicating and the at least one external receiver and transmitter.

In some embodiments, the external receiver is interfaced with a computer terminal or video monitor. In some further embodiments, the computer terminal is a notebook computer. In other embodiments, the external receiver is a tablet or smart phone. In some further embodiments, the tablet or smart phone comprises an application (app) that communicates with the wireless viewing device 300. In still further embodiments, the app that communicates with the wireless viewing device 300 is a dedicated app. In some embodiments, the wireless viewing device 300 is provided with a unique identifier that can be entered into/associated with the app for dedicated communication between an individual wireless viewing device 300 and the app. In some embodiments, the app is capable of recording the transmission from the wireless viewing device as a video or individual pictures/screen captures. In further embodiments, the recordings can be saved into an archive, such as a medical record of the subject. In particular embodiments, the app is capable of remotely controlling functions of the wireless viewing device. For example, functions that could be controlled remotely include, but are not limited to, camera focus, camera optical zoom, camera digital zoom, camera field of view, camera angle, camera rotation, light on/off, and light intensity.

In other embodiments, the housing 310 further comprises a video display showing real time images from a camera 324 of the device.

In some embodiments, the wireless viewing device 300 comprises non-volatile memory for storing images or information from a performed procedure, wherein said images or information can be retrieved from the device. In some embodiments, the non-volatile memory is an RFID tag. In other embodiments, the non-volatile memory is a micro-SD card.

As shown in FIG. 3, in some embodiments, the housing further comprises a power source 316. In some embodiments, the power source 316 is a battery. In further embodiments, the battery is a lithium battery. In some embodiments, the power source 316 is installed within the wireless viewing device 300 upon manufacture, or prior to provision to a practitioner. In other embodiments, the power source 316 is provided separately from the wireless viewing device 300 and installed into the wireless viewing device 300 prior to the use of the wireless viewing device 300 in a procedure. In some embodiments, the power source 316 is removable from the wireless viewing device 300 for separate disposal.

In some embodiments, the power source 316 for the wireless viewing device 300 comprises a power receiver for a radio-frequency (RF)-based power system. The power receiver for an RF-based power system receives energy waveforms from a transmitter and converts the RF-based energy to DC current. In some embodiments, the power receiver comprises at least one power antenna for collecting RF-based energy waveforms from a transmitter. In further embodiments, the receiver comprises multiple power antennas for collecting RF-based energy waveforms from a power transmitter. In still further embodiments, the power receiver comprises paired power antennas for collecting RF-based energy waveforms from a power transmitter. In other still further embodiments, the power receiver comprises at least one power antenna array for collecting RF-based energy waveforms from a power transmitter. In some embodiments, the power receiver is configured to receive and convert energy waveforms from a power transmitter located at least 30 feet away from the wireless viewing device 300. In other embodiments, the power receiver is configured to receive and convert energy waveforms from a power transmitter located at least 15 feet away from the wireless viewing device 300. In still other embodiments, the power receiver is configured to receive and convert energy waveforms from a power transmitter located at least 10 feet away from the wireless viewing device 300. In yet other embodiments, the power receiver is configured to receive and convert energy waveforms from a power transmitter located at least 5 feet away from the wireless viewing device 300. In some embodiments, a power source 316 that comprises a power receiver for an RF-based power system further comprises a battery for storing energy received through and converted by the power receiver. In some embodiments, the battery is a rechargeable battery.

Still referring to FIG. 3, the wireless viewing device 300 comprises a wand 320 that is extendable through an entry portal to a target location in a subject. The wand 320 comprises a proximal end 321 and a distal end 322, wherein the proximal end 321 of the wand 320 is attached to the distal end of the housing 310. In some embodiments, the wand 320 is flexible. In other embodiments, the wand 320 is rigid. In some embodiments, the wand 320 is composed of a clear material. In further embodiments, the clear material is polycarbonate. In some embodiments, the wand 320 is marked with gradations showing how far the wand 320 had been inserted through an entry portal. In some embodiments, the wand 320 is solid. In other embodiments, the wand 320 is tubular with a hollow central lumen. In still other embodiments, the wand 320 is solid, save for channels between the proximal 321 and distal 322 ends of the wand 320 for wiring or optical fibers. In some further embodiments, the channels are internal within the wand 320. In other embodiments, the channels are indents in an external surface of the wand 320.

In some embodiments, at least one surface of the wand 320 is flattened. In other embodiments, the body of the wand 320 is generally rounded, circular, oval or elliptical. In some embodiments, the distal end 322 of the wand 320 is angled between 20 and 70 degrees. In further embodiments, the distal end 322 of the wand 320 is angled between 30 and 60 degrees. In some particular embodiments, the distal end 322 of the wand 320 is angled about 30 degrees. further embodiments, the distal end 322 of the wand 320 is angled between 30 and 60 degrees. In other particular embodiments, the distal end 322 of the wand 320 is angled about 45 degrees. In certain embodiments, the distal end 322 comprises a leading edge that is turned upwards, allowing the wand 320 to separate and form a passage from the entry portal through/between/under/over body tissues to and/or past a target tissue. In some further embodiments, the edge can be flattened.

The wand 320 further comprises a camera 324 affixed at its distal end 322. In some embodiments, the camera 324 has a resolution of at least 100×100 pixels. In a further embodiment, the camera 324 has a resolution of at least 150×150 pixels. In a still further embodiment, the camera 324 has a resolution of at least 200×200 pixels. In an even further embodiment, the camera 324 has a resolution of at least 250×250 pixels. In some embodiments, the camera 324 is a NANEYE camera. In some embodiments, the camera 324 is connected to the internal control board having transmitter functions 314 by wire. In some embodiments, the camera can be rotated, optically zoomed, digitally zoomed and focused either by controls on the wireless viewing device or remotely, such as by an app on a computer, smart phone or tablet. In some embodiments, the light source 312 is located at the distal end 322 of the wand 320 along with the camera 324.

In some embodiments, the wand 320 has a clear covering over the camera 324 and/or light source 312 at the distal end 322 of the wand 320. In some embodiments, the clear covering is polycarbonate. In some embodiments, the clear cover has magnifying properties.

In some embodiments, the camera 324 is located within the housing and comprises an optical fiber component that extends into the wand 320.

In some embodiments, the housing 310 fits in the palm of a hand. In some embodiments, manipulation of the entire wireless viewing device 300 can be done with a single hand.

In an alternative embodiment, the camera 324 remains in a fixed position within the housing 310. In such an embodiment, the camera 324 comprises an image transmitting optical fiber, which is attached at its proximal end to the camera 324, with the distal end of the image transmitting optical fiber being located in the proximity of the distal end 322 of the wand 320, such that from the fixed position of the image transmitting optical fiber the camera 324 can observe and image tissues through the distal end 322 of the wand 320. In another alternative embodiment, the light source 312 also remains in a fixed position within the housing 310. In such an embodiment, the light source 312 comprises a light transmitting optical fiber, which is attached at its proximal end to the light source 312, with the distal end of the light transmitting optical fiber being located in the proximity of the distal end 322 of the wand 320, such that from the fixed position of the light transmitting optical fiber the light source 312 can illuminate tissues through the distal end 322 of the wand 320.

### Kit

Another aspect of the present application relates to an instrument kit for wireless observation of a target tissue within the body of a subject in need thereof. The kit comprises the wireless viewing device of the present application.

In some embodiments, the instrument kit comprises additional components and implements useful for wireless observation of a target tissue within the body of a subject in need thereof.

In some embodiments, the instrument kit comprises a cannula. In further embodiments, the cannula is a slotted cannula. In still further embodiments, the slot extends from the proximal end of the cannula to the proximity of the distal end. In other still further embodiments, the distal end of the cannula is closed. In yet other still further embodiments, the distal end of the cannula is angled upwards. In even other still further embodiments, the distal end of the cannula comprises an edge for separating tissues.

In other further embodiments, the cannula is composed of a clear material, such as polycarbonate. In still further embodiments, the cannula comprises at least one wing, flange or handle at or near the proximal end.

In another embodiment, the instrument kit further includes a scalpel.

In another embodiment, the instrument kit further includes at least one retractor for holding open an entry portal.

In another embodiment, the instrument kit further includes suture material and or at least one bandage.

In another embodiment, the instrument kit comprises a power source for the wireless viewing device of the present application. In a further embodiment, the power source is a battery.

### Method for Endoscopic or Arthroscopic Surgery

Another aspect of the present application relates to a method for uniportal endoscopic or arthroscopic surgery using the wireless viewing device of the present application. Uniportal endoscopic or arthroscopic surgery allows the practitioner to visualize a target tissue and its surrounding tissues as well as perform a surgical procedure through a single entry portal. In some instances, the entry portal may be a natural opening, while in other instances the entry portal is an incision. In the case of an incision, generally only a single small incision must be made. In particular embodiments, the incision is less than or equal to about 2 cm in length. In more particular embodiments, the incision is less than or equal to about 1.5 cm in length. In still more particular embodiments, the incision is less than or equal to about 1 cm in length. The single small incision allows the patient to recover more quickly and begin therapy and/or resume normal activity as tolerated sooner. In some embodiments, the procedure can be a uniportal percutaneous endoscopic surgical procedure.

The uniportal endoscopic surgical procedure described herein can be used to implement a number of different surgical procedures including, but not limited to, carpal tunnel release, Guyon's canal (or canal) release, cubital tunnel release, plantar fascia release, lateral release for patella realignment, release of radial tunnel, release of pronatar teres, release of trigger finger, release of lacertus fibrosus, release of the extensor tendons for lateral epicondylitis, release of medial epicondylitis, release of the posterior and other compartments of the leg, forearm fascia release for fascial compartment syndrome, release of fascial compartments in the upper or lower extremities, relieving the compression of a nerve by a ligament pulley or tunnel, releasing the travel of a ligament through a ligament pulley or tunnel, surgical procedures on the spine, such as endoscopic discectomy for the treatment of degenerative disc disease, herniated discs, bulging discs, pinched nerves or sciatica, endoscopic procedures on cranial and facial tissues, fasciotomy release and blood vessel harvesting.

One embodiment of the present application relates to a method for a performing a uniportal endoscopic or arthroscopic surgical procedure a target tissue in a subject. Generally, the surgical procedure requires the establishment of an entry portal. In some embodiments of the present application, the entry portal is established to the proximate side of the target tissue. In other embodiments of the present application, the entry portal is established to the distal side of the target tissue.

In some embodiments, the establishing an entry portal comprises making an incision.

In some embodiments, following the establishment of an entry portal, the distal end of the cannula portion of the device is inserted through the portal to establish an opening in the underlying tissue between the portal and the target tissue. In some embodiments, the distal end of the cannula portion of the device comprises a front edge for separating tissues. In some embodiments, the cannula is expanded laterally in order to spread the tissue, establishing a passage from the entry portal to the target tissue.

The camera is advanced from the housing and into the cannula to view the target tissue and the surrounding tissues, assuring that the slot of the cannula is in proper orientation to the target tissue.

In one embodiment, the surgical procedure is carpal tunnel release. In a further embodiment, the target tissue is the transverse carpal ligament.

In another particular embodiment, the surgical procedure is trigger finger release. In another particular embodiment, the target tissue is the Al pulley.

### Other Embodiments

One aspect of the present application relates to a wireless viewing device for endoscopic and arthroscopic procedures. The device comprises a housing having a proximal end and a distal end; a wand having a proximal end and a distal end, wherein the proximal end of the wand adjoins the distal end of the housing; a camera with a lens; a light source; a control board having transmitter functions for wirelessly communicating with at least one external receiver; and a power source, wherein the control board and power source are enclosed by the housing and the camera with a lens and the light source are located at or proximate to the distal end of the wand. In some embodiments, the wireless viewing device is insertable into a cannula.

In some embodiments, the device can be handheld. In some further embodiments, the housing is formed in a shape to facilitate gripping with a single hand.

In other embodiments, the device can be held by any other device.

The device can be used on any target tissue, bone, joint or target area of the body of a subject.

In some embodiments, the lens of the camera has a diameter of less than 5 mm.

In other embodiments, the light source is an LED light source.

In still other embodiments, the camera is a NANEYE camera.

Still another aspect of the present application relates to a system for wireless endoscopic and arthroscopic procedures. The system comprises a wireless device and an external receiver/transmitter for visualizing images transmitted by the device. The device comprises a housing having a proximal end and a distal end; a wand having a proximal end and a distal end, wherein the proximal end of the wand adjoins the distal end of the housing; a camera with a lens; a light source; a control board having transmitter functions for wirelessly communicating with at least one external receiver; and a power source, wherein the control board and power source are enclosed by the housing and the camera with a lens and the light source are located at or proximate to the distal end of the wand.

In some embodiments, the device can be handheld. In some further embodiments, the housing is formed in a shape to facilitate gripping with a single hand.

In other embodiments, the device can be held by any other device.

The device and system can be used on any target tissue, bone, joint or target area of the body of a subject.

In some embodiments, the system further comprises a clear cannula that the wand can be inserted into, wherein the cannula comprises a proximal end, a distal end and an open slot extending from the proximal end to the proximity of the distal end.

In still other embodiments of the system, the external wireless receiver/transmitter is selected from the group consisting of video monitors, computer terminals comprising a monitor, smart phones and tablet computers. In some embodiments, function of the tools in the device is controlled with a dedicated application (app) installed or resident on the external wireless receiver/transmitter.

Yet another aspect of the present application relates to a method for performing an endoscopic or arthroscopic procedure with a wireless viewing device. The device comprises a housing having a proximal end and a distal end; a wand having a proximal end and a distal end, wherein the proximal end of the wand adjoins the distal end of the housing; a camera with a lens; a light source; a control board having transmitter functions for wirelessly communicating with at least one external receiver; and a power source, wherein the control board and power source are enclosed by the housing and the camera with a lens and the light source are located at or proximate to the distal end of the wand. The method comprises the steps of: a) establishing an entry portal; b) inserting the distal end of a cannula through the entry portal; c) advancing the cannula to create a passage to a target tissue; d) inserting the distal end of the wand into the proximal end of the cannula and advancing the wand toward the distal end of the cannula; and e) imaging the target tissue with the camera.

In some embodiments, the device can be handheld. In some further embodiments, the housing is formed in a shape to facilitate gripping with a single hand.

In other embodiments, the device can be held by any other device.

The device can be used on any target tissue, bone, joint or target area of the body of a subject.

A further aspect of the present application relates to a kit for performing an endoscopic or arthroscopic procedure. The kit comprises a wireless viewing device for endoscopic and arthroscopic procedures. The device comprises a housing having a proximal end and a distal end; a wand having a proximal end and a distal end, wherein the proximal end of the wand adjoins the distal end of the housing; a camera with a lens; a light source; a control board having transmitter functions for wirelessly communicating with at least one external receiver; and a power source, wherein the control board and power source are enclosed by the housing and the camera with a lens and the light source are located at or proximate to the distal end of the wand.

In some embodiments, the kit further comprises a cannula.

In some embodiments, the device can be handheld. In some further embodiments, the housing is formed in a shape to facilitate gripping with a single hand.

In other embodiments, the device can be held by any other device.

The device can be used on any target tissue, bone, joint or target area of the body of a subject.

The present invention is further illustrated by the following examples which should not be construed as limiting. The contents of all references, patents and published patent applications cited throughout this application, as well as the Figures, are incorporated herein by reference.

### Example 1

### Uniportal Endoscopic Carpal Tunnel Release Using a Wireless Viewing Device with a Slotted Cannula

In a patient presenting with carpal tunnel syndrome, an incision is made just proximal or distal to the carpal transverse ligament, making an entry portal.

The distal end of the cannula of the wireless viewing device is inserted into entry portal and the front edge of the cannula is introduced into the incision and used to create a plane under the carpal transverse ligament, but superficial to the median nerve, with the slot of the cannula facing the carpal transverse ligament. The procedure is observed with the camera. The camera is moved within the cannula to observe and image the target tissue and surrounding tissues.

Following the creation of the plane, the camera is withdrawn back into the housing of the device. The probe along with the camera is then advanced into the cannula to visualize the edges of the transverse carpal ligament. The probe and camera are then withdrawn back into the device.

The blade is then selected and in association with the camera and moved into the cannula. The cutting edge of the blade is moved into contact with the edge of the transverse carpal ligament and the ligament is divided by advancing the cutting edge of the blade through the ligament. The blade is retracted back into the housing of the device.

The camera is moved back into the cannula and the cut edges of the carpal transverse ligament and the underlying median nerve and tendons attached to the digits are visualized.

While visualizing the nerve and tendons, release is confirmed by passive manipulation of the digits through their range of motion.

The cannula is removed from the incision. The device is properly discarded as medical waste.

The wound is closed and a soft bandage is applied. In some cases, a splint is also applied to immobilize the wrist up to a week.

### Example 2

### Endoscopic Visualization of a Target Tissue

In a patient presenting with potential tear of a tendon requiring visual inspection, an incision is made proximate to the proximal or distal edge of the target tendon to establish an entry portal.

A slotted clear cannula is prepared for insertion through the entry portal by introducing an obturator into the lumen of the cannula. A dissector is introduced into the entry portal to form a pathway extending across the target tendon to at least the margin of the tendon distal to the entry portal. Once the pathway is created and the dissector removed, the obturator and the cannula are introduced into the same pathway. Following insertion, the obturator is withdrawn from the lumen of the cannula.

The wireless viewing device is activated and the distal end of the wand is inserted into the cannula. The wand is then advanced distally through the cannula. The camera is used to visually inspect the condition of the tendon and then withdrawn from the cannula. The cannula is then withdrawn from the pathway and entry portal. The wireless viewing device and the cannula are discarded as medical waste.

The wound is closed and a soft bandage is applied.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The aspects and embodiments are intended to cover the components and steps in any sequence which is effective to meet the objectives there intended, unless the context specifically indicates the contrary.

What is claimed is:

1. A wireless viewing device for endoscopic and arthroscopic procedures, comprising;
a housing having a proximal end and a distal end;
a wand having a proximal end and a distal end, wherein the proximal end of the wand adjoins the distal end of the housing;
a camera with a lens;
a light source;
a control board having transmitter functions for wirelessly communicating with at least one external receiver; and
a power source,
wherein the control board and power source are enclosed by the housing, and
wherein the camera with a lens and the light source are located at or proximate to the distal end of the wand.

2. The device of claim 1, wherein the device can be used on any target tissue, bone, joint or target area of the body of a subject.

3. The device of claim 1, wherein the device can be handheld.

4. The device of claim 1, further comprising an antenna.

5. The device of claim 1, further comprising a receiver for receiving wireless communication from an external transmitter.

6. The device of claim 1, wherein the wand is made of a transparent material.

7. The device of claim 1, wherein the distal end of the wand comprises an edge for separating tissues.

8. The device of claim 1, wherein the distal end of the wand is turned upwards like an obturator.

9. The device of claim 1, wherein the lens of the camera has a diameter of less than 5 mm.

10. The device of claim 1, wherein the light source is an LED light source.

11. The device of claim 1, wherein wirelessly communicating is performed by Bluetooth.

12. The device of claim 1, wherein wirelessly communicating is performed by radio frequency.

13. The device of claim 1, wherein wirelessly communicating is performed by WiFi.

14. The device of claim 1, wherein the wand is insertable into a cannula.

15. A wireless device for performing video-assisted endoscopic and arthroscopic procedures, comprising:
a wand having a proximal end and a distal end;
a camera with a lens;
a light source for illuminating a target tissue;
a control board having transmitter and receiver functions for wirelessly communicating with at least one external receiver/transmitter;
an antenna; and
a power source,
wherein the camera is located at the distal end of the wand, and
wherein the control board, light source and antenna are mounted inside an enclosed housing, wherein the housing has a distal end and a proximal end, wherein the wand is attached to the distal end of the housing, and wherein the device further comprises a light-transmitting optical fiber extending from the proximity of the light source to the proximity of the distal end of the wand.

16. The wireless device of claim 15, wherein the device can be used on any target tissue, bone, joint or target area of the body of a subject.

17. The wireless device of claim 15, wherein the device can be handheld.

18. The device of claim 15, further comprising a clear cover over the camera at the distal end of the wand.

19. The device of claim 15, wherein the wand is flexible.

20. A viewing system for wireless endoscopic and arthroscopic procedures, comprising:
(a) a wireless viewing device comprising:
a housing having a proximal end and a distal end;
a wand having a proximal end and a distal end, wherein the proximal end of the wand adjoins the distal end of the housing;
a camera with a lens;
a light source;
a control board having transmitter functions for wirelessly communicating with at least one external receiver; and
a power source,
wherein the control board and power source are enclosed by the housing, and
wherein the camera with a lens and the light source are located at or proximate to the distal end of the wand; and
(b) an external receiver/transmitter for visualizing images transmitted by the device.

21. The system of claim 20, wherein the device can be used on any target tissue, bone, joint or target area of the body of a subject.

22. The system of claim 20, wherein the external wireless receiver/transmitter is selected from the group consisting of video monitors, computer terminals comprising a monitor, smart phones and tablet computers.

23. A method for performing an endoscopic or arthroscopic procedure with a wireless viewing device comprising:
the wireless viewing device comprising:
a housing having a proximal end and a distal end;
a wand having a proximal end and a distal end, wherein the proximal end of the wand adjoins the distal end of the housing;
a camera with a lens;
a light source;
a control board having transmitter functions for wirelessly communicating with at least one external receiver; and
a power source,
wherein the control board and power source are enclosed by the housing, and
wherein the camera with a lens and the light source are located at or proximate to the distal end of the wand;
the method comprising the steps of:
a) establishing an entry portal;
b) inserting the distal end of a cannula through the entry portal;
c) advancing the cannula to create a passage to a target tissue;
d) inserting the distal end of the wand into the proximal end of the cannula and advancing the wand toward the distal end of the cannula; and
e) imaging the target tissue with the camera.

24. The method of claim 23, wherein the device can be used on any target tissue, bone, joint or target area of the body of a subject.

25. A kit for performing an endoscopic or arthroscopic procedure, comprising:
(a) a wireless viewing device comprising:
a housing having a proximal end and a distal end;
a wand having a proximal end and a distal end, wherein the proximal end of the wand adjoins the distal end of the housing;
a camera with a lens;
a light source;
a control board having transmitter functions for wirelessly communicating with at least one external receiver; and
a power source,
wherein the control board and power source are enclosed by the housing, and
wherein the camera with a lens and the light source are located at or proximate to the distal end of the wand; and
(b) instructions.

26. The kit of claim 25, wherein the device can be used on any target tissue, bone, joint or target area of the body of a subject.

27. The kit of claim 25, further comprising a cannula.

28. The kit of claim 25, further comprising an instrument for establishing an entry portal.
